(19) United States
(12) Patent Application Publication
(10) Pub. No.: US 2018/0105882 A1
(43) Pub. Date: Apr. 19, 2018

(54) NEW BIOMARKER FOR OUTCOME IN AML

(71) Applicants: INSERM (INSTITUT NATIONAL DE LA SANTE ET DE LA RECHERCHE MEDIALE), Paris (FR); INSTITUT JEAN PAOLI & IRENE CALMETTES, Marseille (FR); UNIVERSITE D'AIX MARSEILLE; CENTRE ANTIONAL DE LA RECHERCHE SCIENTIFIQUE (CNRS), Paris (FR)

(72) Inventors: Michel AURRAND-LIONS, Marseille Cedex 9 (FR); Maria DE GRANDIS, Marseille Cedex 9 (FR); Norbert VEY, Marseille Cedex 9 (FR)

(21) Appl. No.: 15/562,043

(22) PCT Filed: Mar. 30, 2016
(86) PCT No.: PCT/EP2016/056922

§ 371 Date: Sep. 27, 2017

(30) Foreign Application Priority Data
Mar. 31, 2015 (EP) 15305471.3

(51) Int. Cl. C12Q 1/6886; G01N 33/574

(52) U.S. Cl. CPC C12Q 1/6886; G01N 33/57492; G01N 33/57426; G01N 2800/52; C12Q 2600/118; C12Q 2600/158; G01N 2400/00

(57) ABSTRACT

The present invention relates to a method for predicting the survival time of a patient suffering from acute myeloid leukemia (AML) comprising i) determining the frequency of JAM-C expressing LSCs in a sample obtained from the patient ii) comparing the frequency determined at step i) with its predetermined reference value and iii) providing a good prognosis when the frequency determined at step i) is lower than its predetermined reference value, or 10 providing a bad prognosis when the frequency determined at step i) is higher than its predetermined reference value.

### FIELD OF THE INVENTION

The present invention relates to a method for predicting the survival time of a patient suffering from acute myeloid leukemia (AML) comprising i) determining the frequency of JAM-C expressing LSCs in a sample obtained from the patient ii) comparing the frequency determined at step i) with its predetermined reference value and iii) providing a good prognosis when the frequency determined at step i) is lower than its predetermined reference value, or providing a bad prognosis when the frequency determined at step i) is higher than its predetermined reference value.

### BACKGROUND OF THE INVENTION

Hematopoiesis is the process by which Hematopoietic Stem Cells (HSCs) replenish platelets, red blood cells and immune cells over lifetime. It occurs in the bone marrow (BM) of adult mammals and requires retention of HSCs in specialized “niches” that control HSCs quiescence, proliferation and differentiation into transient amplifying progenitors. Acute Myeloid Leukemia (AML) are hierarchically organized in a similar manner to normal hematopoietic cells. They are characterized by clonal growth of an undifferentiated haematopoietic cell that develops as a consequence of a set of numerous genetic and epigenetic lesions. AML are thought to arise from genetically altered HSCs and progenitors which give rise to fully transformed Leukemic Stem Cell (LSC) in a stepwise manner. LSCs keep self-renewal capacity, pluripotency and quiescence and are functionally defined as a subset of leukemic cells able to transfer AML disease in xenografted mice [Lapidot T et al., 1994]. LSCs are enriched within the CD34+CD38^{Low} fraction of leukemic cells but are also present to a lesser extend in CD34+CD38+ and CD34−CD38− subsets [Bhatia J E et al., 1998; Sarry J E et al., 2011]. They reconstitute the bulk of the tumor once xenografted in recipient mice and are thought to be responsible for disease relapse. This has been supported by a recent study showing that expression of an LSC-related (LSC-R) gene expression signature is associated with poor prognosis in AML [Eppert et al., 2011]. This suggests that intrinsic and extrinsic cues controlling the maintenance of LSCs at a preleukemic stage (pre-LSC) are essential for AML disease evolution [Pandolfi Et al., 2013]. This includes intrinsic properties of pre-LSC and micro-environmental cues provided by stromal cells and immune pressure. Recent progresses in cell sorting by flow cytometry and genome sequencing have allowed studying clonal relationships and genetic alterations in LSCs versus bulk of leukemic cells versus normal hematopoietic cells. Since LSCs are thought to be responsible for relapse, one approach has been to study clonal evolution of AML disease between diagnosis and relapse by whole-genome sequencing [Ding et al., 2012]. Other experimental setups have consisted in gene expression profiling of purified LSCs using phenotypic markers such as CD123 that distinguish healthy HSC from LSC [Eppert et al., 2011; Vergez F et al., 2011; Barreyro et al., 2012]. Although these approaches were highly informative with respect of pathways driving LSC maintenance, none of them tested expression of adhesion molecules involved in bone marrow retention such as JAM-C.

### SUMMARY OF THE INVENTION

In this study, the inventors found that frequencies of JAM-C expressing LSCs in AML is a prognostic marker at diagnosis in AML and that expression of JAM-C is associated with a specific gene signature in the subset of LSCs defined as CD45^{low}CD34⁺CD38^{low/-}CD123⁺CD41^{Neg} leukemic cells.

Thus, the present invention relates to a method for predicting the survival time of a patient suffering from acute myeloid leukemia (AML) comprising i) determining the frequency of JAM-C expressing LSCs in a sample obtained from the patient ii) comparing the frequency determined at step i) with its predetermined reference value and iii) providing a good prognosis when the frequency determined at step i) is lower than its predetermined reference value, or providing a bad prognosis when the frequency determined at step i) is higher than its predetermined reference value.

### DETAILED DESCRIPTION OF THE INVENTION

A first aspect of the invention relates to a method for predicting the survival time of a patient suffering from acute myeloid leukemia (AML) comprising i) determining the frequency of JAM-C expressing LSCs in a sample obtained from the patient ii) comparing the frequency determined at step i) with its predetermined reference value and iii) providing a good prognosis when the frequency determined at step i) is lower than its predetermined reference value, or providing a bad prognosis when the frequency determined at step i) is higher than its predetermined reference value.

In another embodiment, the invention relates to a method for predicting the overall survival (OS) of a patient suffering from acute myeloid leukemia (AML) comprising i) determining the frequency of JAM-C expressing LSCs in a sample obtained from the patient ii) comparing the frequency determined at step i) with its predetermined reference value and iii) providing a good prognosis when the frequency determined at step i) is lower than its predetermined reference value, or providing a bad prognosis when the frequency determined at step i) is higher than its predetermined reference value.

In another embodiment, the invention relates to a method for predicting the leukemia free survival (LFS) of a patient suffering from acute myeloid leukemia (AML) comprising i) determining the frequency of JAM-C expressing LSCs in a sample obtained from the patient ii) comparing the frequency determined at step i) with its predetermined reference value and iii) providing a good prognosis when the frequency determined at step i) is lower than its predetermined reference value, or providing a bad prognosis when the frequency determined at step i) is higher than its predetermined reference value.

As used herein, the term “Overall survival (OS)” denotes the percentage of people in a study or treatment group who are still alive for a certain period of time after they were diagnosed with or started treatment for a disease, such as AML (according to the invention). The overall survival rate is often stated as a five-year survival rate, which is the percentage of people in a study or treatment group who are alive five years after their diagnosis or the start of treatment.

As used herein, the term “Leukemia Free Survival (LFS)”denotes the length of time after primary treatment for a cancer ends that the patient survives without any signs or symptoms of that cancer or without disease progression. End-points will be the date of relapse or death.

As used herein, the term “Good Prognosis” denotes a patient with significantly enhanced probability of survival after disease diagnosis or after treatment inducing complete remission.

As used herein, the term “the frequency of JAM-C expressing LSCs” denotes the number (or the percentage) of LSCs which express JAM-C at their surface. Indeed, according to the invention, the inventors showed that for the outcome of the AML, the number of LSCs which express JAM-C is important. When a patient has a frequency of JAM-C expressing LSCs or a number of LSCs which express JAM-C lower than a predetermined threshold than the prognosis is good and when a patient has a frequency of JAM-C expressing LSCs or a number of LSCs which express JAM-C higher than a predetermined threshold than the prognosis is bad.

Thus, the invention also relates to a method for predicting the survival time of a patient suffering from acute myeloid leukemia (AML) comprising i) determining the number of LSCs which express JAM-C in a sample obtained from the patient ii) comparing the number determined at step i) with its predetermined reference value and iii) providing a good prognosis when the number determined at step i) is lower than its predetermined reference value, or providing a bad prognosis when the number determined at step i) is higher than its predetermined reference value.

As used herein, the term Leukemic Stem Cells (LSC) denotes a population of cells responsible of the initiation and propagation of AML. These cells are characterised by the expression of several markers and thus are CD45^{low}CD34⁻CD38^{low/-}CD123⁺CD41^{Neg}.

Thus, in a particular embodiment, the invention relates to a method for predicting the survival time of a patient suffering from acute myeloid leukemia (AML) comprising i) determining the frequency of JAM-C expressing cells in CD45^{low}CD34⁺CD38^{low/-}CD123⁺CD41^{Neg} leukemic cells obtained from the patient ii) comparing the frequency determined at step i) with its predetermined reference value and iii) providing a good prognosis when the frequency determined at step i) is lower than its predetermined reference value, or providing a bad prognosis when the frequency determined at step i) is higher than its predetermined reference value.

As used herein and according to all aspects of the invention, the term “JAM-C” for “Junctional Adhesion Molecule-C” denotes a protein member of the junctional adhesion molecule protein family and acts as a receptor for another member of this family. An exemplary sequence for human JAM-C protein is deposited in the UniProt database under accession number Q9BX67. The term “JAM-C” also relates to the Junctional Adhesion Molecule-C protein which is encoded by the JAM 3 gene.

The term “JAM 3” has its general meaning in the art and relates to Junctional Adhesion Molecule-3, the gene coding for JAM-C protein.

As used herein and according to all aspects of the invention, the term “sample” denotes, blast cells isolated from bone marrow aspirates, blood, peripheral-blood, purified Hematopoietic Stem Cells (HSC) or purified Leukemic Stem Cells (LSC).

In a further embodiment of the invention, methods of the invention comprise measuring the frequency of JAM-C expressing LSCs of at least one further biomarker or prognostic score.

The term “biomarker”, as used herein, refers generally to a cytogenetic marker, a molecule, the expression of which in a sample from a patient can be detected by standard methods in the art (as well as those disclosed herein), and is predictive or denotes a condition of the subject from which it was obtained.

Various validated prognostic biomarkers or prognostic scores may be combined to JAM-C in order to improve methods of the invention and especially some parameters such as the specificity (see for example Cornelissen et al. 2012).

For example, the other biomarkers may be selected from the group of AML biomarkers consisting of cytogenetics markers (like t(8;21), t(15;17), inv(16) see for example Grimwade et al., 2010or Byrd et al., 2002), lactate dehydrogenase (see for example Haferlach et al 2003), FLT3, NPM1, CEBPα (see for example Schnittger et al., 2002, Dohner et al., 2010). The prognostic scores that may be combined to JAM-C may be for example the Hematopoietic Cell Transplantation Comorbidity Index (HCT-CI) (Sorror et al 2005), the comorbidity and disease status (Sorror et al 2007) or the disease risk index (DRI) (Armand et al 2012).

The inventors also showed that expression of JAM-C is associated with a specific gene signature in LSCs. This signature could be used as prognostic biomarker of AML outcome.

Thus, the invention also relates to a method for predicting the survival time of a patient suffering from acute myeloid leukemia (AML) comprising i) determining in a sample obtained from the patient the expression level on LSCs of at least on gene selected from a first group of genes consisting of: JAM3; AMICA 1; UGT8; CLEC4A; IL23A; BACE2; DPY19L2P2; UNC13; LOC619207; FJX1; SLC44A5; CD226; CHRNA6; CHRNA5; PTPRJ; ADRA2C; NUDT13; HOXB9; HPGD; SLC41A1; RGS1; DEXI; SAMD3; TGM5; IGFBP2; GZMA; DOCK9; LOC100506688; LRRC26; CARD17; FAM159A; SLFN5; RNA5SP96; SOCS1; BMPR1A; IFI30; ADAM19; CORO2A; SLC12A8; MIR221 and the expression level of at least on gene selected from a second group of genes consisting of LXN; HOXA2; HOXA6; HOXA3; CNKSR2; HOXA7; LYZ; PDIA3P; PCTP; TANC1; HOXA4; HLA-DQB1; IL13RA1; MAN1A1; SAV1; ARNTL2; KIF21A; OTTHUMG00; FGD5; AADAT; HIVEP1; ZNF883; LRRC1; HLA-DQA2; NEDD4; CIITA; PAQR5; AAED1; HLA-DPB1; GPSM2; HLA-DPB2; TMEM65; HOXA5; NPR3; SLC9A2; C4orf32; ZNF560; RTKN2; GUCY1B3; NRGN; SPAG1; MICA; HLA-DQA1; GFRA1; HLA-DMB; MFAP2; HLA-DMA; TWSG1; LGALSL; TMOD2; CD74; PIK3C2A; ESAM; LOC728323; FLJ43681; HLA-DRA; ID3; PRRG1; SNAI2; SLC16A9; KLF7; NEUROG3 ii) comparing the expression level of the genes determined at step i) with their predetermined reference values and iii) providing a good prognosis when at least one gene of the second group is higher than its predetermined reference value, or providing a bad prognosis when at least one gene of the first group is higher than its predetermined reference value.

Thus, the invention also relates to a method for predicting the survival time of a patient suffering from acute myeloid leukemia (AML) comprising i) determining the frequency of JAM-C expressing LSCs in a sample obtained from the patient ii) determining in said sample obtained from the patient the expression level on LSCs of at least on gene selected from a first group of genes consisting of: JAM3; AMICA 1; UGT8; CLEC4A; IL23A; BACE2; DPY19L2P2; UNC13; LOC619207; FJX1; SLC44A5; CD226; CHRNA6; CHRNA5; PTPRJ; ADRA2C; NUDT13; HOXB9; HPGD; SLC41A1; RGS1; DEXI; SAMD3; TGM5; IGFBP2; GZMA; DOCK9; LOC100506688; LRRC26; CARD17; FAM159A; SLFN5; RNA5SP96; SOCS1; BMPR1A; IFI30; ADAM19; CORO2A; SLC12A8; MIR221 and the expression level of at least on gene selected from a second group of genes consisting of LXN; HOXA2; HOXA6; HOXA3; CNKSR2; HOXA7; LYZ; PDIA3P; PCTP; TANC1; HOXA4; HLA-DQB1; IL13RA1; MAN1A1; SAV1; ARNTL2; KIF21A; OTTHUMG00; FGD5; AADAT; HIVEP1; ZNF883; LRRC1; HLA-DQA2; NEDD4; CIITA; PAQR5; AAED1; HLA-DPB1; GPSM2; HLA-DPB2; TMEM65; HOXA5; NPR3; SLC9A2; C4orf32; ZNF560; RTKN2; GUCY1B3; NRGN; SPAG1; MICA; HLA-DQA1; GFRA1; HLA-DMB; MFAP2; HLA-DMA; TWSG1; LGALSL; TMOD2; CD74; PIK3C2A; ESAM; LOC728323; FLJ43681; HLA-DRA; ID3; PRRG1; SNAI2; SLC16A9; KLF7; NEUROG3 iii) comparing the frequency determined at step i) with its predetermined reference value and comparing the expression level of the genes determined at step i) with their predetermined reference values and iv) providing a good prognosis when the frequency determined at step i) is lower than its predetermined reference value and/or when at least one gene of the second group is higher than its predetermined reference value, or providing a bad prognosis when the frequency determined at step i) is higher than its predetermined reference value and/or when at least one gene of the first group is higher than its predetermined reference value.

The present invention also relates to JAM-C as a biomarker for outcome of AML patients.

Measuring the frequency of JAM-C expressing LSCs (or the number of LSCs which express JAM-C) can be done by measuring the gene expression level of JAM-C or by measuring the level of the protein JAM-C and can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies. Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino -3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can he produced that are identifiable based on their different spectral characteristics. For example, semiconductor nanocrystals can he produced that emit light of different colors hased on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlsbad, Calif.).

Additional labels include, for example, radioisotopes (such as 3 H), metal chelates such as DOTA and DPTA chelates of radioactive or paramagnetic metal ions like Gd3+, and liposomes.

Detectable labels that can he used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can he used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn)). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6× SCC. SCC is a 0.15 M NaC1, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from the sample of the invention and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes such as the actin gene ACTB, ribosomal 18S gene, GUSB, PGK1, TFRC, GAPDH, GUSB, TBP and ABL1. This normalization allows the comparison of the expression level in one sample, e.g., a patient sample, to another sample, or between samples from different sources.

According to the invention, measuring the level of the protein JAM-C may also be measured and can be performed by a variety of techniques well known in the art.

Typically, levels of protein expression defining expressing and non-expressing cells may be measured for example by flow cytometry, capillary electrophoresis-mass spectroscopy technique (CE-MS) or ELISA performed on the sample. In a particular embodiment, the PrimeFlow™ RNA assay may be used (see PrimeFlow™ RNA Assay User Manual and Protocol by Affymetric Ebioscience).

In the present application, the “level of protein” or the “protein level expression” means the quantity or concentration of said protein. In another embodiment, the “level of protein” means the level of JAM-C protein fragments. In still another embodiment, the “level of protein” means the quantitative measurement of the protein JAM-C expression relative to a negative control.

Such methods comprise contacting a sample with a binding partner capable of selectively interacting with proteins present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS).etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

Methods of the invention may comprise a step consisting of comparing the proteins and fragments concentration in circulating cells with a control value. As used herein, “concentration of protein” refers to an amount or a concentration of a transcription product, for instance the protein JAM-C. Typically, a level of a protein can be expressed as nanograms per microgram of tissue or nanograms per milliliter of a culture medium, for example. Alternatively, relative units can be employed to describe a concentration. In a particular embodiment, “concentration of proteins” may refer to fragments of the protein JAM-C. Thus, in a particular embodiment, fragment of JAM-C protein may also be measured.

In a particular embodiment, the detection of the frequency of JAM-C expressing LSCs can be performed by flow cytometry. When this method is used, the method consists of determining the frequency of JAM-C expressing LSC and particularly in the subset CD45^{low}CD34⁺CD38^{low/-}CD123⁺CD41^{Neg} leukemic cells. According to the invention and the flow cytometry method, when the florescence intensity is high or bright, LSCs are JAM-C Positive (POS) and thus the expression level of JAM-C is high and when the florescence intensity is low or dull, LSCs are JAM-C negative (NEG) and thus the expression level of JAM-C is low.

Thus, according to the invention when the frequency of JAM-C expressing LSCs is high the prognosis of the patient suffering from AML is bad and when the frequency of JAM-C expressing LSCs is low the prognosis of the patient suffering from AML is good.

Predetermined reference values used for comparison may comprise “cut-off” or “threshold” values that may be determined as described herein. Each reference (“cut-off”) value for JAM-C expression may be predetermined by carrying out a method comprising the steps of

a) providing a collection of samples from patients suffering of AML;

b) determining the frequency of JAM-C expressing cells for each sample contained in the collection provided at step a);

c) ranking the tumor tissue samples according to said frequency

d) classifying said samples in pairs of subsets of increasing, respectively decreasing, number of members ranked according to the frequency,

e) providing, for each sample provided at step a), information relating to the actual clinical outcome for the corresponding cancer patient (i.e. the duration of the leukemia free survival (LFS) or the overall survival (OS) or both);

f) for each pair of subsets of samples, obtaining a Kaplan Meier percentage of survival curve;

g) for each pair of subsets of samples calculating the statistical significance (p value) between both subsets

h) selecting as reference value for the frequency of JAM-C expressing LSCs , the value of frequency for which the p value is the smallest.

For example the frequency of JAM-C expressing LSCs has been assessed for 100 AML samples of 100 patients. The 100 samples are ranked according to their frequencies. Sample 1 has the highest frequency and sample 100 has the lowest frequency. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding AML patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the frequency corresponding to the boundary between both subsets for which the p value is minimum is considered as the reference value. It should be noted that the reference value is not necessarily the median value of frequencies.

In routine work, the reference value (cut-off value) may be used in the present method to discriminate AML samples and therefore the corresponding patients.

Kaplan-Meier curves of percentage of survival as a function of time are commonly used to measure the fraction of patients living for a certain amount of time after treatment and are well known by the man skilled in the art.

The man skilled in the art also understands that assessment of the expression level of a gene in individual cells or limited number of cells should of course be used for obtaining the reference value and thereafter for assessment of frequencies or percentages of cells expressing a gene in patient samples subjected to the method of the invention.

According to the invention, the inventors have determined a threshold value of 0.4%. This value signifies that when a patient has more than 0.4% of LSC positive for JAM-C (high % JAM-C) then he has a bad prognosis and when a patient has less than 0.4% of LSC positive for JAM-C (low % JAM-C) then he has a good prognosis.

As used herein the term “a patient with more than 0.4% of LSC positive for JAM-C” denotes that more than 0.4% of the LSCs of the patient express JAM-C at their surface.

As used herein the term “a patient with less than 0.4% of LSC positive for JAM-C” denotes that less than 0.4% of the LSCs of the patient express JAM-C at their surface.

According to the invention, this threshold value of 0.4% may be applied to the subset of LSCs defined as CD45^{low}CD34⁺CD38^{low/-}CD123⁺CD41^{Neg} leukemic cells.

A further object of the invention relates to kits for performing the methods of the invention, wherein said kits comprise means for measuring the frequency of JAM-C expressing LSCs in the sample obtained from the patient.

The kits may include probes, primers macroarrays or microarrays as above described. For example, the kit may comprise a set of probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kit of the invention may comprise amplification primers that may be pre-labelled or may contain an affinity purification or attachment moiety. The kit may further comprise amplification reagents and also other suitably packaged reagents and materials needed for the particular amplification protocol.

Therapeutic Methods

In another object, the invention relates to an inhibitor of JAM-C or an inhibitor of the JAM-C gene expression for use in the treatment of acute myeloid leukemia (AML) in patient with a bad prognosis as described above.

According to the invention, a patient with a higher frequency of JAM-C than its predetermined reference values as described above is eligible for a treatment using an inhibitor of JAM-C or an inhibitor of the JAM-C gene expression according to the invention.

Thus, the methods of the invention as described above may be useful to determine the eligibility of a patient to be treated with inhibitor of JAM-C or an inhibitor of the JAM-C gene expression.

According to the invention, the methods of the invention may be used as a companion diagnostic under a treatment of patient affected with AML.

The inventors show that JAM-C expressing cells are endowed with malignant properties such as increased clonogenic and leukemic potentials.

Thus, the invention also relates to an inhibitor of JAM-C or an inhibitor of the JAM-C gene expression for use in the treatment of acute myeloid leukemia (AML) in patient with a bad prognosis as described above wherein JAM-C is expressed at the surface of LSCs of the patient.

In other word, the invention relates to an inhibitor of JAM-C expressed at the surface of the LSCs of the patient wherein the inhibitor may be an inhibitor of JAM-C or an inhibitor of the JAM-C gene expression for use in the treatment of acute myeloid leukemia (AML) in patient with a bad prognosis as described above.

In a particular embodiment, the invention relates to an inhibitor of JAM-C or an inhibitor of the JAM-C gene expression for use in the treatment of acute myeloid leukemia (AML).

In another particular embodiment, the invention also relates to an inhibitor of JAM-C or an inhibitor of the JAM-C gene expression for use in the treatment of acute myeloid leukemia (AML) wherein JAM-C is expressed at the surface of LSCs of the patient.

In other word, the invention also relates to an inhibitor of JAM-C expressed at the surface of the LSCs of the patient wherein the inhibitor may be an inhibitor of JAM-C or an inhibitor of the JAM-C gene expression for use in the treatment of acute myeloid leukemia (AML).

As used herein, the term “inhibitor” denotes a molecule which can inhibit the activity of the protein (e.g. inhibit the junctional adhesion function of the protein) or a molecule which destabilizes the protein.

In one embodiment, the LSCs are CD45^{low}CD34⁺CD38^{low/-}CD123⁺CD41^{Neg} cells.

Typically, the inhibitor according to the invention includes but is not limited to a small organic molecule, an antibody, and a polypeptide.

In one embodiment, the inhibitor according to the invention may be a low molecular weight compound, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

In one embodiment, the compound according to the invention is an antibody. Antibodies directed against the JAM-C protein can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against JAM-C protein can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-JAM-C protein single chain antibodies. Compounds useful in practicing the present invention also include anti-JAM-C protein, antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to JAM-C protein.

Humanized anti-JAM-C protein antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies of JAM-C protein are selected.

In a particular embodiment, the antibody according to the invention may be an antibody as described in the patent applications WO2008038127 or WO2005/050213.

According to the invention, the JAM-C antibody may be coupled with a toxin and thus directly target the LSCs to kill these cells thank to the ADC properties (Antibody-drug Conjugates) and thus treat AML. In this case, the couple antibody and toxin is called an immunotoxin.

As used herein, the term ‘immunotoxin” has its general meaning in the art. By “immunotoxin”, it is meant a chimeric protein made of an antibody or modified antibody or antibody fragment (also called in the present application “antibody”), attached to a fragment of a toxin. The antibody of the immunotoxin is covalently attached to the fragment of a toxin. Preferably, the fragment of the toxin is linked by a linker to the antibody or fragment thereof. Said linker is preferably chosen from 4-mercaptovaleric acid and 6-maleimidocaproic acid.

The anti-JAM-C immunotoxin also comprises a toxin or a fragment thereof. Particularly, said toxin or its fragment is a Ribosome Inactivating Protein (RIP).

Particularly, the Ribosome Inactivating Protein is chosen from saporin, ricin, abrin, gelonin, Pseudomonas exotoxin (or exotoxin A), trichosanthin, luffin, agglutinin and the diphtheria toxin.

Particularly, the toxin may also be a chemical drug.

Particularly, the toxin is chosen from modeccin, mitogellin, chlortetracycline, mertansine, monomethyl auristatin E, monomethyl auristatin F, and enediynes, especially calicheamicins (like calicheamicin k or calicheamicin γl) and their related esperamicins (like esperamicin A1). Enediynes are chemical compounds characterized by either 9- or 10-membered rings containing two triple bonds separated by a double bond.

When the toxin is mertansine, it is linked to the antibody or a fragment thereof by a linker. When the linker is 4-mercaptovaleric acid, the group comprising the toxin and the linker is called emtansine.

When the toxin is monomethyl auristatin E (MMAE), it is linked to the antibody or a fragment thereof by a structure comprising a spacer (which is preferably paraaminobenzoic acid), a cathepsin-cleavable linker (preferably consisting of citrulline and valine) and an attachment group or linker (preferably consisting of 6-maleimidocaproic acid). Preferably in such a case, the group comprising the toxin and the structure as defined in the previous sentence is vedotin.

When the toxin is monomethyl auristatin F (MMAF), it is linked to the antibody or a fragment thereof by a structure comprising an attachment group or linker (preferably consisting of 6-maleimidocaproic acid). Preferably in such a case, the group comprising the toxin and the structure as defined in the previous sentence is mafodotin.

The toxin may also be chosen from anticancer agents. Said anticancer agents are preferably chosen from combrestatin, colchicine, actinomycine, duocarmycins and their synthetic analogues (adozelesin, bizelesin and carzelesin), fludarabine, gemcitabine, capecitabine, methotrexate, taxol, taxotere, mercaptopurine, thioguanine, hydroxyurea, cytarabine, cyclophosphamide, ifosfamide, platinum complexes (such as cisplatin, carboplatin and oxaliplatin), mitomycin, dacarbazine, procarbizine, etoposide, teniposide, campathecins, bleomycin, doxorubicin, idarubicin, daunorubicin, dactinomycin, plicamycin, mitoxantrone, L-asparaginase, epimbicin, 5-fluorouracil, taxanes (such as docetaxel and paclitaxel), leucovorin, levamisole, irinotecan, estramustine, etoposide, nitrogen mustards, BCNU, nitrosoureas (such as carmustine and lomustine), vinca alkaloids (such as vinblastine, vincristine, dolastatins and vinorelbine), imatinib mesylate, hexamethylenediamine, topotecan, kinase inhibitors (like the tyrosine kinase inhibitors called tyrphostins), phosphatase inhibitors, ATPase inhibitors, protease inhibitors, inhibitors of herbimycin A, genistein, erbstatin, and lavendustin A.

The toxin may also be a radioisotope, preferably chosen from ²¹¹At, ¹³¹I, ¹²⁵I, ¹⁸⁶Re, ¹⁸⁸Re, ¹⁵³Sm, P³², ⁹⁰Y, ¹⁷⁷Lu, ⁶⁷Cu, ⁴⁷Sc, ²¹²Bi, ²¹³Bi, ²²⁶Th, ¹¹¹In and ⁶⁷Ga.

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of JAM-C protein are selected.

In one embodiment, the compound according to the invention is a polypeptide or a peptide.

In a particular embodiment the polypeptide is a functional equivalent of JAM-C protein. As used herein, a “functional equivalent” of JAM-C is a compound which is capable of binding to JAM-C, thereby preventing the interaction of JAM-C with its molecular partners. The term “functional equivalent” includes fragments and mutants of JAM-C. The term “functionally equivalent” thus includes any equivalent of JAM-C obtained by altering the amino acid sequence, for example by one or more amino acid deletions, substitutions or additions such that the protein analogue retains the ability to bind to these proteins. Amino acid substitutions may be made, for example, by point mutation of the DNA encoding the amino acid sequence.

The polypeptides of the invention may be produced by any suitable means, as will be apparent to those of skill in the art. In order to produce sufficient amounts of Jam-C protein or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the compound according to the invention is an inhibitor of JAM-C gene expression.

Small inhibitory RNAs (siRNAs) can also function as inhibitors of JAM-C expression for use in the present invention. JAM-C gene expression can be reduced by contacting a subject or cell with a small double stranded RNA (dsRNA), or a vector or construct causing the production of a small double stranded RNA, such that JAM-C gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of JAM-C gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of JAM-C mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of JAM-C gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing JAM-C. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991.

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and micro encapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

In another object, the invention relates to an inhibitor of at least on gene selected from the group of genes consisting of: JAM3; AMICA 1; UGT8; CLEC4A; IL23A; BACE2; DPY19L2P2; UNC13; LOC619207; FJX1; SLC44A5; CD226; CHRNA6; CHRNA5; PTPRJ; ADRA2C; NUDT13; HOXB9; HPGD; SLC41A1; RGS1; DEXI; SAMD3; TGM5; IGFBP2; GZMA; DOCK9; LOC100506688; LRRC26; CARD17; FAM159A; SLFN5; RNA5SP96; SOCS1; BMPR1A; IFI30; ADAM19; CORO2A; SLC12A8; MIR221 for use in the treatment of acute myeloid leukemia (AML) in patient with a bad prognosis as described above.

According to the invention, patients with overexpression of at least one gene of the list above than its predetermined reference values are eligible for a treatment using an inhibitor of these genes according to the invention.

Thus, the methods of the invention as described above may be useful to determine the eligibility of a patient to be treated with inhibitor of at least one gene of the list above.

As used herein, the term “inhibitor” denotes a molecule which can inhibit the activity of the gene or the related protein or a molecule that inhibit the gene expression.

Typically, the inhibitor of the genes according to the invention includes but is not limited to a small organic molecule, an antibody, and a polypeptide.

Therapeutic Composition

A third object of the invention relates to a pharmaceutical composition comprising an inhibitor of JAM-C or an inhibitor of the JAM-C gene expression, for use in the treatment of acute myeloid leukemia (AML) in patient with a bad prognosis as described above.

In another embodiment, the invention relates to a pharmaceutical composition comprising an inhibitor of JAM-C or an inhibitor of the JAM-C gene expression for use in the treatment of acute myeloid leukemia (AML) wherein JAM-C is expressed at the surface of LSCs.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment. In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising a compound according to the invention and a further therapeutic active agent.

For example, anti-cancer agents may be added to the pharmaceutical composition as described below.

Anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca²⁺ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazo lam.

Screening Method

In a further aspect, the present invention relates to a method of screening a candidate compound for use in the treatment of acute myeloid leukemia (AML) in patient with a bad prognosis as described above, wherein the method comprises the steps of: i) providing candidate compounds and ii) selecting candidate compounds that inhibit JAM-C or JAM-C gene expression.

In a further aspect, the present invention relates to a method of screening a candidate compound for use in the treatment of acute myeloid leukemia (AML) in a subject in need thereof, wherein the method comprises the steps of:

providing a cell, tissue sample or organism expressing JAM-C,

providing a candidate compound such as small organic molecule, antibodies, peptide or polypeptide,

measuring the activity of JAM-C,

and selecting positively candidate compounds that inhibit JAM-C.

Methods for measuring the inhibition of JAM-C are well known in the art. For example, inhibition of JAM-C expressing LSCs adhesion to bone marrow stromal cells could be used as described in Arcangeli ML et al, 2014. Alternative methods include preclinical models of xenograft in immune-deficient mice (Donate C et al, Canc Res 2013).

Tests and assays for screening and determining whether a candidate compound is a JAM-C inhibitor are well known in the art. In vitro and in vivo assays may be used to assess the potency and selectivity of the candidate compounds to inhibit JAM-C.

The invention will be further illustrated by the following figures and examples. However, these examples and figures should not be interpreted in any way as limiting the scope of the present invention.

### FIGURES

FIG. 1: JAM-C expression at diagnosis and relapse in de novo AML patients

A, B, C. Representative flow cytometry profiles obtained on three different frozen de novo AML patient samples at diagnosis. The gating strategy consisted in selection of single living cells, gated on low expressing CD45 cells and further selected for CD34 expression, low expression of CD38, high expression of CD123. Indicated percentages represent the fraction of CD45^{Low}CD34^{Pos}CD38^{Low/-}CD123^{Pos}CD41^{Neg} expressing JAM-C. D, E, F. Left panels: Representative flow cytometry profiles in paired AML patient samples at diagnosis and relapse. Right panel: Paired analysis of ten paired AML patient samples at diagnosis and relapse. The percentages of JAM-C expressing cells within the CD45^{Low}CD34^{Pos}CD38^{Low/-}CD123^{Pos}CD41^{Neg} population are shown. ***p value<0, 001 in paired t-test.

FIG. 2: Frequencies of JAM-C expressing cells have a prognostic value in de novo AML.

A. Frequency distribution and curve fitting of patient population. 59 frozen samples were analyzed by flow cytometry and the number of samples was plotted against the frequency distribution of JAM-C expressing LSCs with steps of 0.25% frequencies from CD45^{Low}CD34^{Pos}CD38^{Low/-}CD123^{Pos}CD41^{Neg} population. A bimodal distribution is observed. Curve fitting reveals a threshold value of 0.4% between the two groups. B. Kaplan-Meier survival curves of overall survival (OS) of patients with de novo AML are plotted. Gray solid lines (low % JAMC+, n=28) represent OS of patients with frequencies of JAM-C expressing LSCs below 0.4% as defined in A. Black solid lines (high % JAMC+, n=31) represent OS of patients with frequencies of JAM-C expressing LSCs above 0.4%. Median survival are respectively of 4.44 and 1.03 years for the two groups. C. Kaplan-Meier survival curves of Leukemia Free Survival (LFS) of patients with de novo AML are plotted. Gray solid lines (low % JAMC+) represent LFS of patients with frequencies of JAM-C expressing LSCs below 0.4% as defined in A. Black solid lines (high % JAMC+) represent LFS of patients with frequencies of JAM-C expressing LSCs above 0.4%.

FIG. 3: JAM-C expressing LSCs are endowed with functional properties of Leukemic Initiating Cells.

A, B, C. Representative results obtained in Cobblestone Area Forming Cells assays (CAFC) using CD45^{Low}CD34^{Pos}CD38^{Low/-}CD123^{Pos}CD41^{Neg} sorted cells with the indicated phenotype with respect to JAM-C expression. Three representative patients are shown. D. Graphs showing the percentages of chimerism in the blood and bone marrow (BM) of mice xenografted with 350 CD45^{Low}CD34^{Pos}CD38^{Low/-}CD123^{Pos}CD41^{Neg} JAM-C^{Neg} (white bars) or JAM-C^{Pos} sorted cells (black bars). n=3.*p value<0.05.

FIG. 4: Characterization of JAM-C-expressing LSCs isolated from KG1 cell line

A. Clonogenic properties of KG1 variant cell lines in colony forming unit (CFU) assays. Results obtained with CD45^{Low}CD34^{Pos}CD38^{Low/-}CD123^{Pos}CD41^{Neg} JAM-C^{Neg} (white bars) and JAM-CPos (black bars) variant KG1 cell lines are shown. n=4.***p value<0.001. B. Engraftment properties of CD45^{Low}CD34^{Pos}CD38^{Low/-}CD123^{Pos}CD41^{Neg} JAM-C^{Neg (KG}1 JAM-C⁻) and JAM-C^{Pos} (KG1 JAM-C⁺) variant KG1 cell lines.

TABLE 1
List of genes differentially expressed between JAM-C^{Neg} (KG1 JAM-C⁻) and JAM-C^{Pos}
(KG1 JAM-C⁺) variant KG1 cell lines.

| GENES UP-REGULATED IN KG1 | | GENES UP-REGULATED IN KG1 | |
|---|---|---|---|
| JAM-C+ | | JAM-C− | |
| Genes | Public RefSEQ | Genes | Public RefSEQ |
| JAM3 | NM_032801 | LXN | NM_020169 |
| AMICA 1 | NM_001098526 | HOXA2 | ENST00000222718 |
| UGT8 | ENST00000310836 | HOXA6 | ENST00000222728 |
| CLEC4A | NM_016184 | HOXA3 | NM_153631 |
| IL23A | NM_016584 | CNKSR2 | NM_014927 |
| BACE2 | NM_012105 | HOXA7 | NM_006896 |
| DPY19L2 | NM_173812 | LYZ | ENST00000261267 |
| UNC13C | NM_001080534 | PDIA3P | NR_002305 |
| LOC619207 | AY567967 | PCTP | NM_021213 |
| FJX1 | HGNC: HGNC: 17166 | TANC1 | NM_033394 |
| SLC44A5 | ENST00000370859 | HOXA4 | ENST00000360046 |
| CD226 | NM_006566 | HLA-DQB1 | NM_002123 |
| CHRNA6 | NM_004198 | IL13RA1 | NM_001560 |
| CHRNA5 | NM_000745 | MAN1A1 | NM_005907 |
| PTPRJ | NM_002843 | SAV1 | NM_021818 |
| ADRA2C | NM_000683 | ARNTL2 | NM_020183 |
| NUDT13 | NM_015901.5 | KIF21A | NM_001173464 |
| HOXB9 | ENST00000311177 | OTTHUMG00 | OTTHUMG00000158558 |
| HPGD | NM_000860 | FGD5 | NM_152536 |
| SLC41A1 | ENST00000367137 | AADAT | NM_016228 |
| RGS1 | NM_002922 | HIVEP1 | ENST00000379388 |
| DEXI | AF108145.2 | ZNF883 | NM_001101338 |
| SAMD3 | NM_001258275.2 | LRRC1 | NM_018214 |
| TGM5 | NM_201631 | HLA-DQA2 | ENST00000374940 |
| IGFBP2 | NM_000597 | NEDD4 | NM_006154 |
| GZMA | NM_006144 | CIITA | ENST00000324288 |
| DOCK9 | NM_001130048 | PAQR5 | NM_017705 |
| LOC100506688 | NR_104614.1 | AAED1 | ENST00000375234 |
| LRRC26 | ENST00000371542 | HLA-DPB1 | NM_002121 |
| CARD17 | NM_001007232 | GPSM2 | NM_013296 |
| FAM159A | NM_001042693 | HLA-DPB2 | NR_001435 |
| SLFN5 | NM_144975 | TMEM65 | KJ903907.1 |
| RNA5SP96 | NG_033462 | HOXA5 | NM_019102 |
| SOCS1 | NM_003745 | NPR3 | NM_001204375 |
| BMPR1A | NM_004329 | SLC9A2 | NM_003048 |
| IFI30 | NM_006332 | C4orf32 | KJ900147.1 |
| ADAM19 | NM_033274 | ZNF560 | NM_152476 |
| CORO2A | NM_003389 | RTKN2 | NM_145307 |
| SLC12A8 | NM_024628 | GUCY1B3 | NM_000857 |
| MIR221 | NR_029635 | NRGN | NM_006176 |
| | | SPAG1 | NM_003114 |
| | | MICA | NM_001289153.1 |
| | | HLA-DQA1 | CR450297.1 |
| | | GFRA1 | AF038421.1 |
| | | HLA-DMB | NM_002118 |
| | | MFAP2 | NM_017459 |
| | | HLA-DMA | NM_006120 |
| | | TWSG1 | NM_020648.5 |
| | | LGALSL | NM_014181 |
| | | TMOD2 | NM_014548.3 |
| | | CD74 | NM_001025159 |
| | | PIK3C2A | NM_002645 |
| | | ESAM | NM_138961 |
| | | LOC728323 | NR_024437 |
| | | FLJ43681 | NT_010663.16 |
| | | HLA-DRA | NM_019111 |
| | | ID3 | NM_002167 |
| | | PRRG1 | NM_000950 |
| | | SNAI2 | NM_003068 |
| | | SLC16A9 | NM_194298.2 |
| | | KLF7 | NM_003709.3 |
| | | NEUROG3 | NM_020999 |

### EXAMPLE

Material & Methods

Flow Cytometry

Blast cells from peripheral blood of AML patients were collected and frozen at diagnosis and also at relapse from some patients. After thawing, primary AML blast cells were stained with the following conjugated antibodies: CD45-APC-Cy7 (BD Pharmingen), CD34-PC7 (BD Pharmingen), CD38-eFluor450 (eBioscience), CD123-FITC (BD Pharmingen), CD41-PE (Beckman-Coulter), JAM-C-APC (R&D System) or corresponding isotypic control and fixable viability dye-eFluor506 (eBioscience) in accordance with the manufacturer's instructions. Leukocytes were used to define the threshold for positive-staining cells. Analyses were performed on an LSR Fortessa flow cytometer and sorting are performed on FACS ARIAII or ARIA SORP cell sorter.

Survival Analysis

The best threshold for continuous variable was calculated using population frequency analysis. Overall survival (OS) and leukemia-free survival (LFS) rates were measured from the date of diagnosis until death and from the date of complete remission until death or relapse, respectively. Overall and leukemia-free survival rates were were estimated by the Kaplan-Meier method and compared using the log-rank test with GraphPad Prism software.

Clonogenic Assays

MS-5 mouse bone marrow-derived stromal cells were plated in 96-well format (20,000 per well in IMEM containing 10% FBS) and kept at 37° C./5% CO2. One day later, purified viable CD45^{low}/CD34⁻/CD38^{low}/CD123⁺/CD41⁻/JAM-C positive or negative cells from 3 AML patients were added in 100 μL of fresh co-culturing media in each well. 30 (when possible) or 15, 10, 5 and 1 cells were seeded respectively in 12 wells. Six days later, the wells were rinsed and fresh media was added. The co-cultures were then maintained with one subsequent half media change at every weeks and assessed for cobblestones at 5 weeks. A cobblestone was defined as an instance of at least 6 tightly packed cells beneath the MS-5 stromal monolayer. The co-culture media formulation consisted of a-Eagle minimum essential medium, 12.5% fetal bovine serum, 12.5% horse serum, 200 mM glutamine, 1 mM monothioglycerol, 1 μM hydrocortisone and 20 ng/ml human recombinant IL-3. The Colony Forming Unit (CFU) assay was performed to analyse KG1 cells. 103 isolated CD45^{low}/CD34⁺/CD38^{low}/CD123⁺/CD41⁻/JAM-C positive or negative KG1 cells were seeded in methylcellulose medium H4230 (Stem Cell) following the manufacturer's instructions. Quadruplicate cultures were incubated at 37° C. in 5% CO2 and colonies (>20 cells) were scored after 14 days.

Assessment of Engraftment Potential

Isolated CD45^{low}/CD34⁺/CD38^{low/Neg}/CD123⁺/CD41⁻/JAM-C positive or negative cells from AML patient or KG1 were screened to assess whether they had the potential to engraft NOD scid gamma (NSG) mice. 350 AML patient cells or 2×105 KG1 cells were injected through the retro-orbital vein into each mouse previously sublethally irradiated. Mice were sacrificed 20 weeks after graft if injected with AML patient cells or 21 days after graft if injected with KG1 cells. In blood and bone marrow, frequencies of human CD45 positive cells was analysed by flow cytometry.

Affymetrix Array Analysis

Six samples, 3 KG1 JAM-C⁻ and 3 KG1 JAM-C⁺, were processed for microarray analysis using the human gene 2.0 ST chips. Differential gene expression was evaluated using GENE-E.

Statistical Analysis

Statistical significance was determined with nonparametric Mann-Whitney or paired t-tests using Prism software.

Results

JAM-C is Expressed by a Subset of Leukemic Stem Cells in AML

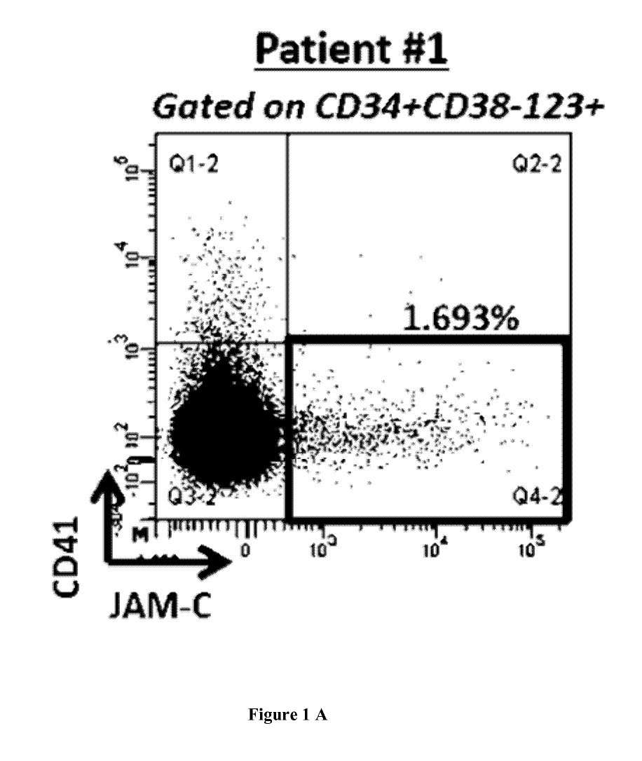

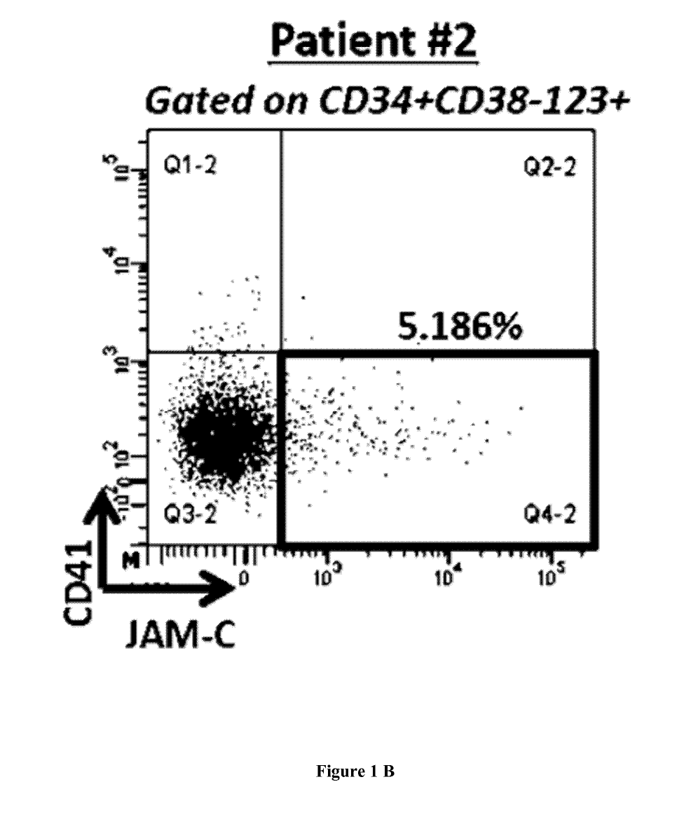

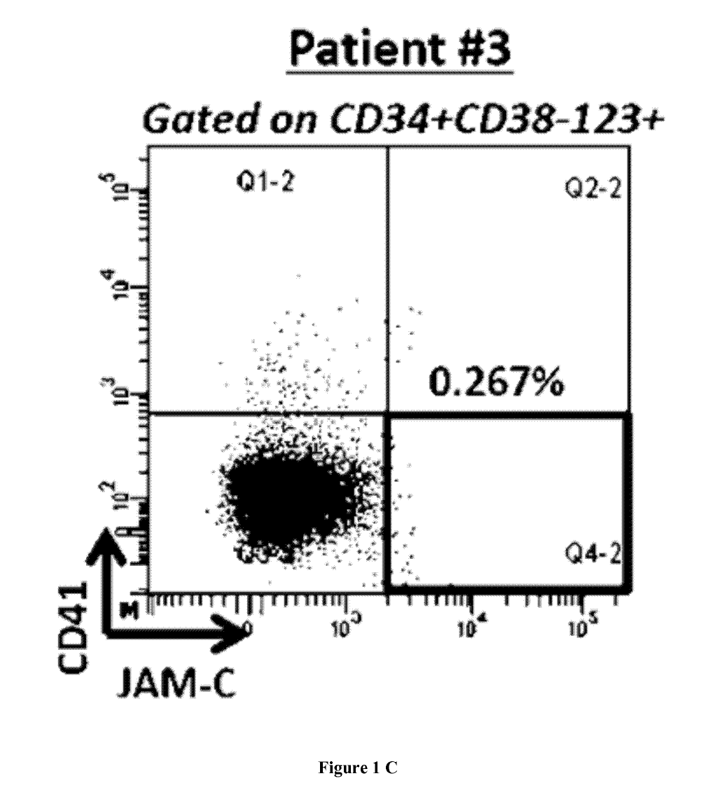

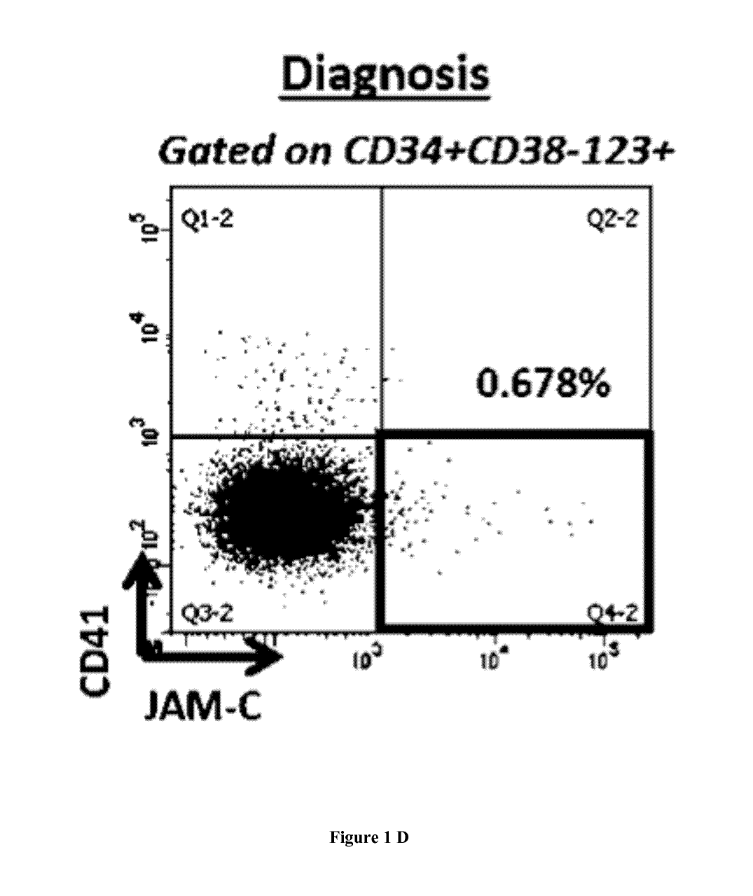

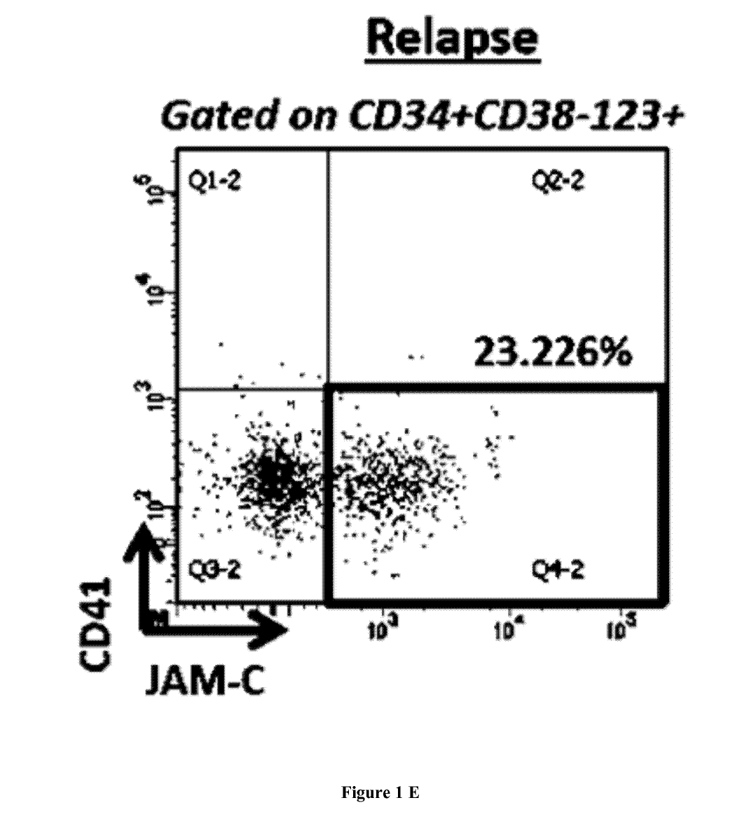

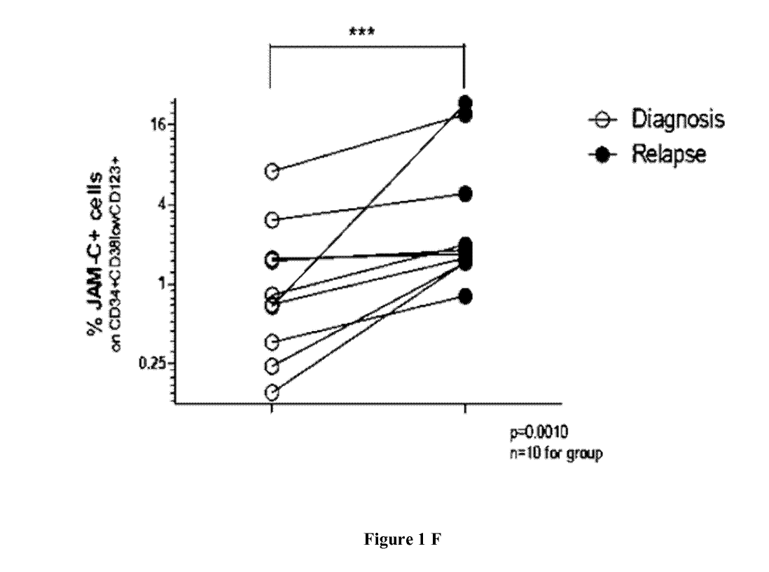

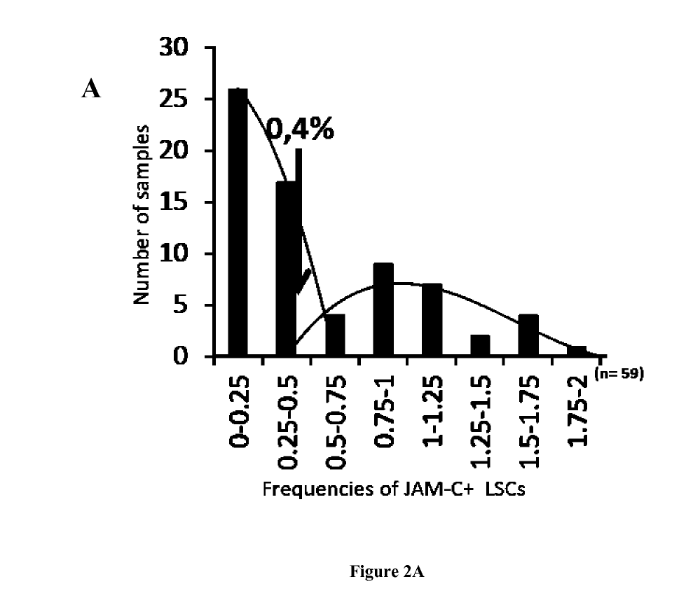

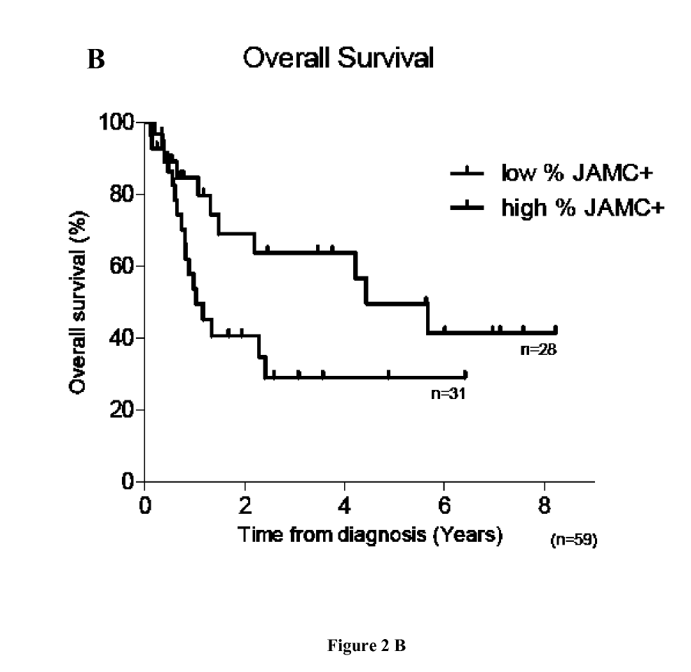

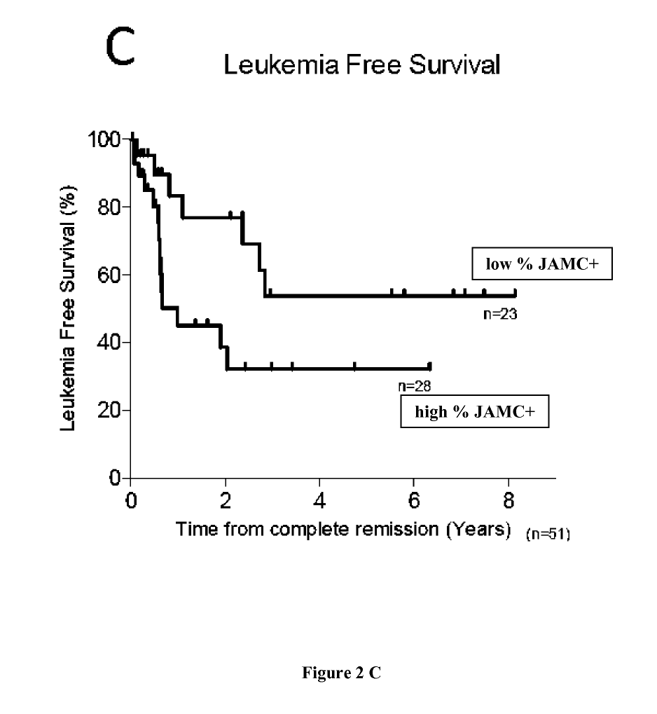

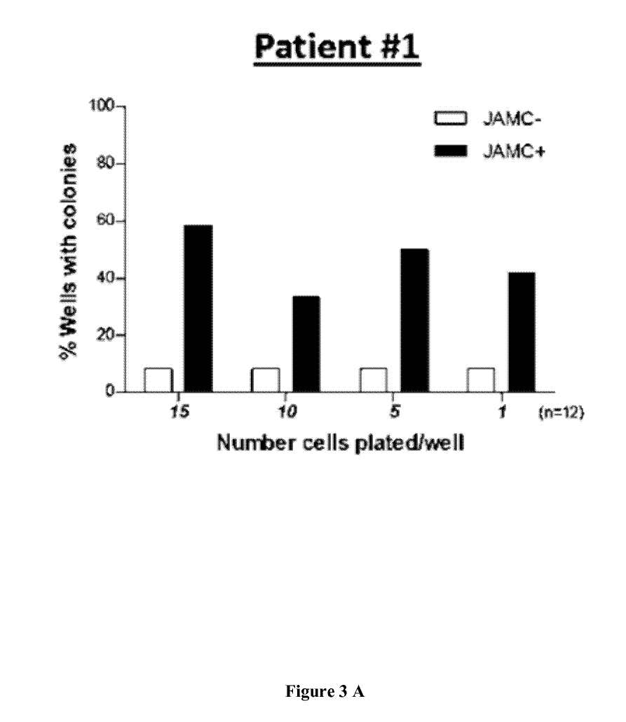

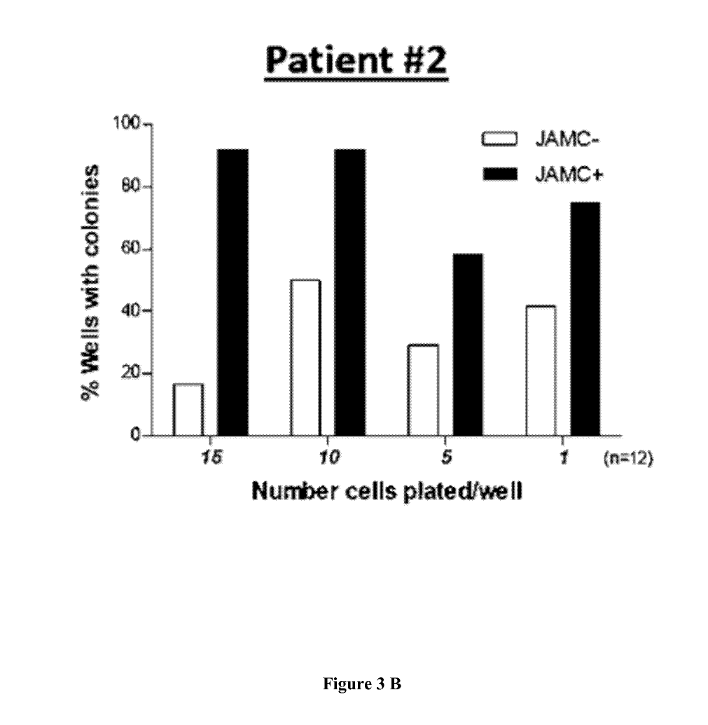

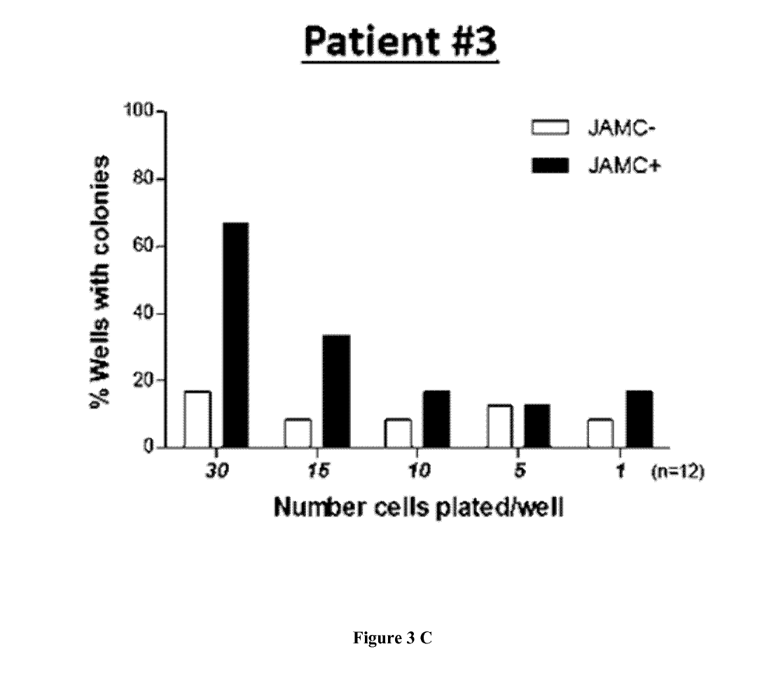

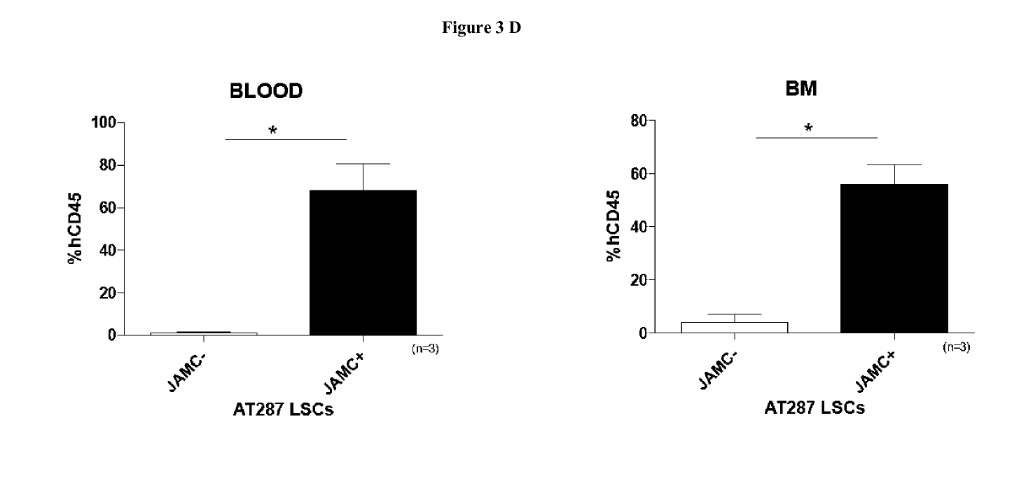

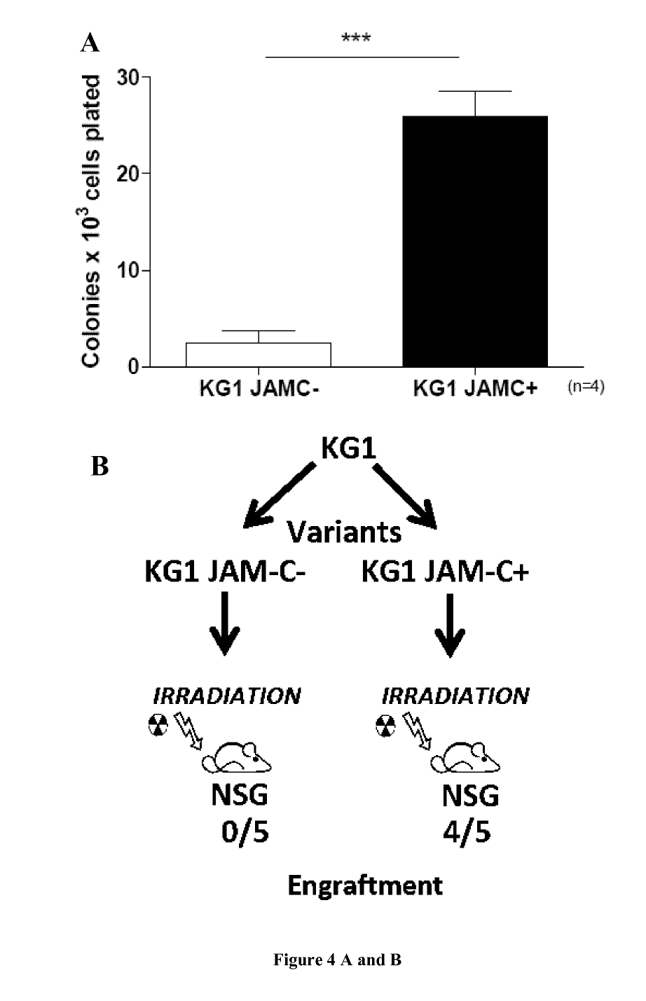

We have recently found that the Junctional Adhesion Molecule-C (JAM-C) contributes to maintenance of HSC quiescence through its interaction with JAM-B expressed by stromal cells [Arcangeli ML et al., 2011; Arcangeli ML et al., 2014]. Since LSCs represent the malignant counterpart of normal HSCs, we thus tested if JAM-C was expressed by leukemic subsets in de novo AML at diagnosis. To this end, we used a previously described flow cytometry strategy [Vergez F et al., 2011 and Barreyro L et al., 2012] to which we added JAM-C and CD41. The latter marker was used to exclude the possibility that JAM-C staining was due to platelets remnants stacked on leukemic cells. We found JAM-C expression on a fraction of LSC, characterized as CD45^{Low}CD34^{Pos}CD38^{Low/-}CD123^{Pos} cells (FIGS. 1 A, B, C). JAM-C and CD41 staining were mutually exclusive indicating that JAM-C expression was not due to platelets sticking on leukemic cells. When paired AML patient samples at diagnosis and relapse were analyzed, we found a significant increase in the frequencies of LSCs expressing JAM-C at relapse (FIGS. 1D, E, F), indicating that treatment selects JAM-C-expressing cells and that JAM-C may be used as a biomarker of clonal disease evolution in AML.

JAM-C Expression Predicts Poor Disease Outcome in De Novo AML

To better understand if JAM-C may be a prognostic marker, frequencies of LSC/JAM-C-expressing cells was analyzed in a cohort of 59 de novo AML patient samples. This revealed a bimodal distribution of LSC/JAM-CPos frequencies that allowed us to define a threshold value of 0.4% using curve fitting and population frequencies (FIG. 2A). Analysis of AML patients overall survival with low versus high LSC/JAM-CPos frequencies showed that high levels of JAM-C expressing cells were associated with inferior overall survival (median 1.03 versus 4.44 years) (FIG. 2B). This was correlated with reduced leukemic free survival in the group of patients presenting LSC/JAM-C^{Pos} frequencies above 0.4% of CD45^{Low}CD34^{Pos}CD38^{Low/-}CD123^{Pos}CD41^{Neg} living cells (FIG. 2C). Since LSCs are endowed with clonogenic and engraftment properties, we then purified JAM-C^{Pos} and JAM-C^{Neg} LSCs and tested their functional properties. Using three different patient samples, we found that JAM-C-expressing cells have an increased ability to form colonies in cobblestone area forming cell assays (CAFC) as compared to LSCs lacking JAM-C expression (FIGS. 3A, B, C). More interestingly, we found that the property to reconstitute the tumor bulk once xenografted in recipient mice was restricted to JAM-C expressing patient's LSCs when limited numbers of cells (350) were xenografted in mice. This indicates that the leukemic initiating activity is highly enriched in JAM-C⁻ expressing subset of LSCs and that genetic and metabolic alterations in these cells are sufficient to give rise to full blown acute myeloid leukemia.

Characterization of JAM-C-expressing LSCs

Since JAM-C expressing LSCs in de novo AML patient samples are rare cells (less than 1500/patient sample), we searched for a cell line that would present similar heterogeneity to de novo AML patient samples. We found that the KG1 cell line was heterogeneous and that a fraction CD34^{Pos}CD38^{Low}CD123^{Pos}KG1 cells (KG1/LSC) expressed JAM-C. We sorted JAM-C^{Pos} and JAM-C^{Neg} KG1/LSC fractions (referred as KG1^{JCPos} and KG1^{JCNeg}) and established two variant cell lines with stable phenotype over at least four weeks in culture (data not shown). These model cell lines were then used to perform functional assays such as engraftment and clonogenic assays. We found that clonogenic activity was highly enriched in the JAM-C positive fraction (FIG. 4A). In addition, engraftment in NSG mice indicated that LSC engraftment was dependent on JAM-C expression (FIG. 4B). Since such properties are the hallmark of leukemic stem and initiating cells, we further performed gene expression profiling of KG1^{JCPos} versus KG1^{JCNeg}. 102 genes encoding essentially transcription factors, adhesion molecules, chemokine receptors and MHC Class I and Class II molecules were differentially expressed between KG1^{JCPos} and KG1^{JCNeg} cells (Table 1). We propose that this gene signature associated to JAM-C expression by LSCs is characteristic of Leukemic Stem Cells endowed with chemotherapeutic resistance in de novo AML and could be used as prognostic biomarker of disease outcome. Not only JAM-C but also all other genes identified as JAM-C+ LSC indirect markers by gene expression analysis could have prognostic value in AML.

### REFERENCES

Throughout this application, various references describe the state of the art to which this invention pertains. The disclosures of these references are hereby incorporated by reference into the present disclosure.

Arcangeli M L, Frontera V, Bardin F, Obrados E, Adams S, Chabannon C, et al. JAM-B regulates maintenance of hematopoietic stem cells in the bone marrow. Blood 2011;118(17):4609-19.

Arcangeli M L, Bardin F, Frontera V, Bidaut G, Obrados E, Adams R H, et al. Function of Jam-B/Jam-C interaction in homing and mobilization of human and mouse hematopoietic stem and progenitor cells. Stem Cells 2014;32(4):1043-54.

Barreyro L, Will B, Bartholdy B, Zhou L, Todorova T I, Stanley R F, et al. Overexpression of IL-1 receptor accessory protein in stem and progenitor cells and outcome correlation in AML and MDS. Blood 2012;120(6):1290-8.

Bhatia M, Bonnet D, Murdoch B, Gan O I, Dick J E. A newly discovered class of human hematopoietic cells with SCID-repopulating activity. Nat Med 1998;4(9):1038-45.

Ding L, Ley T J, Larson D E, Miller C A, Koboldt D C, Welch J S, et al. Clonal evolution in relapsed acute myeloid leukaemia revealed by whole-genome sequencing. Nature 2012;481(7382):506-10.

Doñate C, Ody C, McKee T, Ruault-Jungblut S, Fischer N, Ropraz P, Imhof B A, Matthes T. Homing of human B cells to lymphoid organs and B-cell lymphoma engraftment are controlled by cell adhesion molecule JAM-C. Cancer Res. 2013 Jan 15;73(2):640-51. doi: 10.1158/0008-5472.CAN-12-1756. Epub 2012 Dec. 5.

Eppert K, Takenaka K, Lechman E R, Waldron L, Nilsson B, van G P, et al. Stem cell gene expression programs influence clinical outcome in human leukemia. Nat Med 2011;17(9):1086-93.

Lapidot T, Sirard C, Vormoor J, Murdoch B, Hoang T, Caceres-Cortes J, et al. A cell initiating human acute myeloid leukaemia after transplantation into SCID mice. Nature 1994;367(6464):645-8.

Pandolfi A, Barreyro L, Steidl U. Concise review: preleukemic stem cells: molecular biology and clinical implications of the precursors to leukemia stem cells. Stem Cells Transl Med 2013;2(2):143-50.

Sarry J E, Murphy K, Perry R, Sanchez P V, Secreto A, Keefer C, et al. Human acute myelogenous leukemia stem cells are rare and heterogeneous when assayed in NOD/SCID/IL2Rgammac-deficient mice. J Clin Invest 2011;121(1):384-95.

Vergez F, Green A S, Tamburini J, Sarry J E, Gaillard B, Cornillet-Lefebvre P, et al. High levels of CD34+CD38low/−CD123+blasts are predictive of an adverse outcome in acute myeloid leukemia: a Groupe Ouest-Est des Leucemies Aigues et Maladies du Sang (GOELAMS) study. Haematologica 2011;96(12):1792-8.

1. A method for predicting the survival time of a patient suffering from acute myeloid leukemia (AML) comprising i) determining the frequency of JAM-C expressing Leukemic Stem Cells (LSCs) in a sample obtained from the patient ii) comparing the frequency determined at step i) with its predetermined reference value and iii) providing a good prognosis when the frequency determined at step i) is lower than its predetermined reference value, or providing a bad prognosis when the frequency determined at step i) is higher than its predetermined reference value.

2. A method for predicting the survival time of a patient suffering from acute myeloid leukemia (AML) comprising i) determining the frequency of JAM-C expressing LSCs in a sample obtained from the patient ii) determining in said sample obtained from the patient the expression level on LSCs of at least e one gene selected from a first group of genes consisting of: JAM3; AMICA 1; UGT8; CLEC4A; IL23A; BACE2; DPY19L2P2; UNC13; LOC619207; FJX1; SLC44A5; CD226; CHRNA6; CHRNA5; PTPRJ; ADRA2C; NUDT13; HOXB9; HPGD; SLC41A1; RGS1; DEXI; SAMD3; TGM5; IGFBP2; GZMA; DOCK9; LOC100506688; LRRC26; CARD17; FAM159A; SLFN5; RNA5SP96; SOCS1; BMPR1A; IFI30; ADAM19; CORO2A; SLC12A8; and MIR221 and the expression level of at least one gene selected from a second group of genes consisting of LXN; HOXA2; HOXA6; HOXA3; CNKSR2; HOXA7; LYZ; PDIA3P; PCTP; TANC1; HOXA4; HLA-DQB; IL13RA1; MAN1A1; SAV1; ARNTL2; KIF21A; OTTHUMG00; FGD5; AADAT; HIVEP1; ZNF883; LRRC1; HLA-DQA2; NEDD4; CIITA; PAQR5; AAED1; HLA-DPB1; GPSM2; HLA-DPB2; TMEM65; HOXA5; NPR3; SLC9A2; C4orf32; ZNF560; RTKN2; GUCY1B3; NRGN; SPAG1; MICA; HLA-DQA1; GFRA1; HLA-DMB; MFAP2; HLA-DMA; TWSG1; LGALSL; TMOD2; CD74; PIK3C2A; ESAM; LOC728323; FLJ43681; HLA-DRA; ID3; PRRG1; SNA12; SLC16A9; KLF7; and NEUROG3 iii) comparing the frequency determined at step i) with its predetermined reference value and comparing the expression level of the genes determined at step i) with their predetermined reference values arid iv) providing a good prognosis when the frequency determined at step i) is lower than its predetermined reference value and/or when at least one gene of the second group is higher than its predetermined reference value, or providing a bad prognosis when the frequency determined at step i) is higher than its predetermined reference value and/or when at least one gene of the first group is higher than its predetermined reference value.

3. A method for predicting the survival time of a patient suffering from acute myeloid leukemia (AML) comprising i) determining in a sample obtained from the patient the expression level on LSCs of at least one gene selected from a first group of genes consisting of: JAM3; AMICA 1; UGT8; CLEC4A; IL23A; BACE2; DPY19L2P2; UNC13; LOC619207; FJX1; SLC44A5; CD226; CHRNA6; CHRNA5; PTPRJ; ADRA2C; NUDT13; HOXB9; HPGD; SLC41A1; RGS1; DEXI; SAMD3; TGM5; IGFBP2; GZMA; DOCK9, LOC100506688; LRRC26; CARD17; FAM159A; SLFN5; RNA5SP96; SOCS1; BMPR1A; IF130; ADAM19; CORO2A; SLC12A8; and MIR221 and the expression level of at least one gene selected from a second group of genes consisting of LXN; HOXA2; HOXA6; HOXA3; CNKSR2; HOXA7; LYZ; PDIA3P; PCTP; TANC1; HOXA4; HLA-DQB1; IL13RA1; MAN1A1; SAV1; ARNTL2; KIF21A; OTTHUMG00; FGD5; AADAT; HIVEP1; ZNF883; LRRC1; HLA-DQA2; NEDD4; CIITA; PAQR5; AAED1; HLA-DPB1; GPSM2; HLA-DPB2; TMEM65; HOXA5; NPR3; SLC9A2; C4orf32; ZNF560; RTKN2; GUCY1B3; NRGN; SPAG1; MICA; HLA-DQA1; GFRA1; HLA-DMB; MFAP2; HLA-DMA; TWSG1; LGALSL; TMOD2; CD74; PIK3C2A; ESAM; LOC728323; FLJ43681; HLA-DRA; ID3; PRRG1; SNAI2; SLC16A9; KLF7; and NEUROG3 ii) comparing the expression level of the genes determined at step i) with their predetermined reference values and iii) providing a good prognosis when at least one gene of the second group is higher than its predetermined reference value, or providing a bad prognosis when at least one gene of the first group is higher than its predetermined reference value.

4. The method according to claim 1 wherein the sample is blast cells isolated from bone marrow aspirates, blood, peripheral-blood, purified Hematopoietic Stem Cells (HSC) or purified Leukemic Stem Cells (LSC).

5. A kit for performing the method of claim 1, wherein said kit comprises means for measuring the frequency of JAM-C expressing LSCs in the sample obtained from the patient.

6. (cancel)

7. A method for treating acute myeloid leukemia (AML) in a patient with a bad prognosis as determined in claim 1, comprising the step of administering a therapeutically effective amount of an inhibitor of JAM-C or an inhibitor of the JAM-C gene expression.

8. A method for treating acute myeloid leukemia (AML) in a patient with a bad prognosis as determined in claim 2, comprising the step of administering a therapeutically effective amount of an inhibitor of JAM-C or an inhibitor of the JAM-C gene expression.

9. A method for treating acute myeloid leukemia (AML) in a patient with a bad prognosis as determined in claim 3, comprising the step of administering a therapeutically effective amount of an inhibitor of JAM-C or an inhibitor of the JAM-C gene expression.
